(19) United States
(12) Patent Application Publication
(10) Pub. No.: US 2011/0112282 A1
(43) Pub. Date: May 12, 2011

(54) IDO INHIBITORS AND THERAPEUTIC USES THEREOF

(76) Inventors: Ute Roehrig, Lausanne (CH); Loay Awad, Lausanne (CH); Olivier Michelin, Lausanne (CH); Benoit Van Den Eynde, Brussels (BE); Luc Pilotte, Brussels (BE); Vincent Stroobant, Brussels (BE); Pierre Larrieu, Brussels (BE)

(21) Appl. No.: 12/736,526

(22) PCT Filed: Apr. 15, 2009
(86) PCT No.: PCT/EP2009/054482

§ 371 Date: Jan. 19, 2011

(30) Foreign Application Priority Data
Apr. 15, 2008 (GB) 0806794.4

(51) Int. Cl. C07H 7/04; C07D 209/48; C07C 211/58; C07C 211/63; C07D 215/38; C07C 237/20; C07D 207/16

(52) U.S. Cl. 536/18.7; 548/477; 564/428; 564/391; 564/287; 546/171; 564/194; 548/537

(57) ABSTRACT

Compounds of formula (I), and pharmaceutically acceptable salts thereof, in which each compound is adapted to occupy the binding site of human IDO, which comprises a large hydrophobic pocket A and a second, proximal hydrophobic pocket B, the compound comprising at least one of the following elements: (i) a large hydrophobic fragment to substantially fill pocket A in the binding site of human IDO; (ii) an atom that can coordinate to the heme iron of human IDO, (iii) a positively charged group that can form a salt-bridge with the heme 7-propionate of the human IDO; (iv) a negatively charged group that can form a salt-bridge with Arg231 of the human IDO; (v) a hydrophobic group that can form van der Waals interactions with pocket B; and (vi) one or more substituents that can hydrogen bond to Ser167 and to Gly262, and as IDO inhibitors and their therapeutic use, eg in the treatment of cancer.

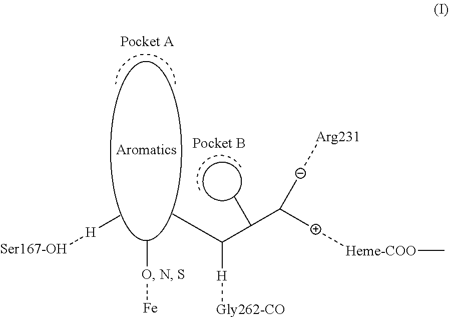

This invention relates to inhibitors of indoleamine 2,3-dioxygenase (IDO) and their use in the treatment of cancer or infections, either alone or in combination with additional therapeutic agents.

The heme-containing enzyme indoleamine 2,3-dioxygenase (IDO, EC 1.13.11.52) has been implicated in the establishment of pathological immune tolerance by tumors. IDO catalyzes the initial and rate-limiting step in the catabolism of tryptophan (Trp) along the kynurenine pathway. By depleting Trp locally, IDO blocks the proliferation of T lymphocytes, which are extremely sensitive to Trp shortage. The observation that many human tumors constitutively express IDO introduced the hypothesis that its inhibition could enhance the effectiveness of cancer immunotherapy. Results from in vitro and in vivo studies suggest that the efficacy of therapeutic vaccination of cancer patients may indeed be improved by concomitant administration of an IDO inhibitor.

Most known IDO inhibitors display affinities in the micromolar range, but recently some submicromolar inhibitors have been discovered. The crystal structures of human IDO [Sugimoto, H.; Oda, S.; Otsuki, T.; Hino, T.; Yoshida, T.; Shiro, Y. Proc Natl Acad Sci USA, 2006, 103(8), 2611-2616] can serve as a scaffold for the in silico design of new and more potent IDO inhibitors.

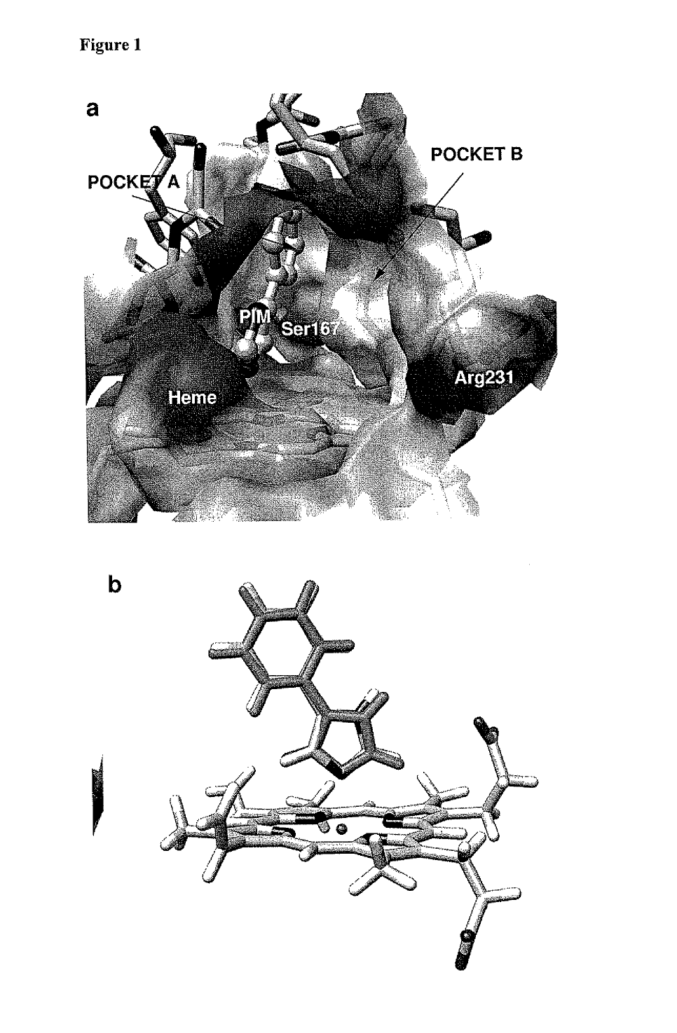

In one X-ray structure of IDO, published by Sugimoto, the inhibitor 4-phenylimidazole (PIM) is bound in a deep binding site, with its phenyl ring inside a large hydrophobic pocket (Pocket A, FIG. 1a). The imidazole nitrogen is coordinated to the heme iron with a distance of 2.1 Å. A second proximal hydrophobic pocket is not occupied by PIM but could interact with larger ligands (Pocket B, FIG. 1).

In FIG. 1, (a) is a representation of an X-ray structure of IDO: Binding site with bound PIM ligand. Two hydrophobic pockets and some important residues are labeled. The surface is coloured by its electrostatic potential (red=negative, blue=positive). (b) is a superposition of PIM crystal structure (green) and best predicted structure from EADock.

We first investigated the binding modes of known IDO inhibitors using our docking algorithm EADock [Grosdidier, A.; Zoete, V.; Michielin, O. Proteins, 2007, 67(4), 1010-1025.]. We find very good agreement between the X-ray structure of PIM and the best binding mode obtained from EADock calculations (RMSD 0.2 Å, FIG. 1b), suggesting that our docking algorithm is capable of finding and correctly ranking the conformation of IDO ligands.

Based on the observed geometries of the bound ligands, we conclude that a good ligand should display some or all of the following features: (i) a large hydrophobic fragment to fill pocket A in the binding site; (ii) an atom that can coordinate to the heme iron such as oxygen, nitrogen, sulphur; (iii) a positively charged group that can form a salt-bridge with the heme 7-propionate; (iv) a negatively charged group that can form a salt-bridge with Arg231; (v) a hydrophobic group that can form van der Waals interactions with pocket B; and (vi) groups that can hydrogen bond to Ser167 and to Gly262.

According to the invention, we provide compounds of formula I, and pharmaceutically acceptable salts thereof, in which each compound is adapted to occupy the binding site of human IDO, which comprises a large hydrophobic pocket A and a second, proximal hydrophobic pocket B, the compound comprising at least one of the following elements:

(i) a large hydrophobic fragment to substantially fill pocket A in the binding site of human IDO;
(ii) an atom that can coordinate to the heme iron of human IDO,
(iii) a positively charged group that can form a salt-bridge with the heme 7-propionate of the human IDO;
(iv) a negatively charged group that can form a salt-bridge with Arg231 of the human IDO;
(v) a hydrophobic group that can form van der Waals interactions with pocket B; and
(vi) one or more substituents that can hydrogen bond to Ser167 and to Gly262.

The occupancy of pocket A by the large hydrophobic fragment may be at least as large as that of PIM. We prefer hydrophobic fragments which are complementary in shape to pocket A, for examples as determined by shape complementarity analysis. Shape complementarity analysis may be carried out using the program SC (http://www.ccp4.ac.uk/ccp4i_main.html). We particularly prefer compounds of formula I that bind to pocket A of human IDO with a good shape complementarity, that is with a S_{c} greater than 0.50, more preferably greater than 0.55, particularly greater than 0.60.

Suitable large hydrophobic fragments adapted to fill pocket A include mono- and bicyclic 5-12 membered aromatic rings. These may be aromatic hydrocarbons, such as benzene and naphthalene, or heterocyclic, such as pyridine or quinoline, for example 1, 2 or 3 quinoline or benzothiazoles. The aromatic rings may be substituted or unsubstituted. When substituted, they may have more than one substituents, e.g. lower alkyl, halogen, etc, provided that this does not prevent the fragment from occupying pocket A.

Suitable atoms that can coordinate to the heme iron of human IDO include nitrogen, oxygen or sulphur. The coordinating atom may be part of the ring making up the large hydrophobic fragment. Alternatively, the coordinating atom may be a substituent on the hydrophobic fragment, for example a substituent containing a hydroxyl group, an amino group, a nitro group, an SH group, an Salkyl group, etc. Compounds of formula I may contain more than one heme coordinating groups.

Positively charged groups that can form a salt-bridge with the heme 7-propionate of the human IDO including protonated amino groups and quaternary ammonium groups, guanadines, and the like.

Negatively charged groups that can form a salt-bridge with Arg231 of the human IDO include carboxylate, sulphate and sulphonate groups.

Hydrophobic group that can form van der Waals interactions with pocket B include lower alkyl groups, e.g. C1-C10, more preferably C1 to C6, hydrocarbon groups, which may be branched, cyclic or linear, saturated or unsaturated.

The one or more substituents that can hydrogen bond to Ser167 and to Gly262 include substituents that are well known accept from and/or donate hydrogen bonds to suitably placed oxygen and hydrogens in amide functions and in hydroxyl groups. Hydrogen bond donating groups include amino, hydroxyl and the hydrogen of a primary or secondary amide. Suitable hydrogen bond accepting groups include oxygen atoms in hydroxy, carbonyl and amide groups and nitrogens, particularly sp₂ hybridised nitrogens, e.g. in imines, and in aromatic heterocyclic rings.

Particular classes of compounds of formula I that satisfy these criteria include quinolines, benzothiazoles, phenylthiazoles, phthalamides and brassinin derivatives

In general compounds of formula I may take the form

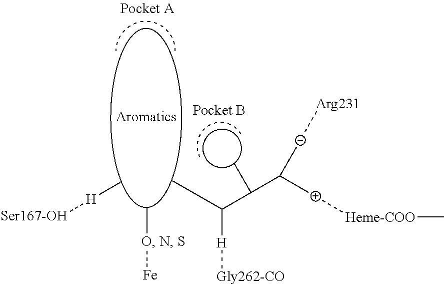

We prefer compounds which have at least two of the features (i) to (vi), more preferably three of the features (i) to (vi), more preferably four of the features (i) to (vi), even more preferably five of the features (i) to (vi), and especially all six of the features (i) to (vi).

Specific compounds of formula I may be devised easily by persons skilled in the art, such as a competent post doctoral medicinal chemist. Fit of molecules can be determined using the known docking programs, for example those mentioned herein.

The compounds of formula I can readily by synthesised, in multistep syntheses, from commercially available starting materials and conventional methods known per se. Textbooks with which the skilled person would be expected to be conversant include Advanced Organic Chemistry by Jerry March and Advanced Practical Organic Chemistry by J. Leonard, B. Lygo, and G. Procter.

Particularly preferred compounds of formula I, together with their IC₅₀, are shown with the Examples.

A preferred group of compounds of formula I are those of formula II

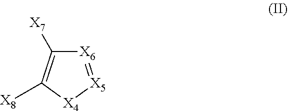

in which
X₄ represents NR¹¹ or S, wherein R¹¹ represents H, pyridyl or phenyl optionally substituted by —OH;
X₅ represents N or CR¹², wherein R¹² represents H, NH₂ or SR₁₃ and R₁₃ represents H or CH₂N(CH₃)₂;
X₆ represents N or CR¹⁴, wherein R¹⁴ represents H or (CH₂)ₚNHC(S)S(CH₂)_{q}OH in which p and q, which may be the same or different, represent an integer from 1-4 inclusive;
either one of X₇ and X₈ represents pyridyl, CH₂C(O)OCH₃ or phenyl optionally substituted by —OH, and the other of X₇ and X₈ represents H, or
X₇ and X₈, together with the carbon atoms to which they are attached form a benzene ring which is optionally substituted by NO₂ or chlorine,
and pharmaceutically acceptable salts thereof.

A group of preferred compounds of formula II is that in which X₄ represents NR¹¹, both X₅ and X₆ represents N.

A further group of preferred compounds of formula II is that in which X₄ represents S, X₅ represents C—R¹² and X₆ represents N, particularly when R₁₂ represents SH, NH₂ or CH₂N(CH₃)₂.

A yet further group of preferred compound of formula II is that in which X₄ represents NH, X₅ represents CH and X₆ represents R¹⁴, particularly (CH₂)ₚNHC(S)S(CH₂)₂OH, in which p is 1 or 2 and

Related molecules that fall within the scope of formula I that may be specifically mentioned are the phthalimides with the following structure:

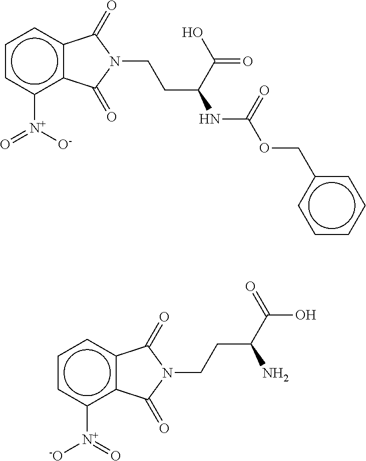

Some of the compounds of formula I may already be known as such, although not previously described for use as a medicine. Accordingly, we provide the compounds of formula I for use as pharmaceuticals.

A preferred group of compounds of formula I are those of formula III,

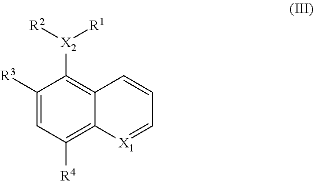

in which
X₁ represents N or C,
X₂ represents H, N or O, provided that when X2 represents H, R1 and R2 have no value and that when X₂ represents O, R₂ has no value,
R₁ represents H, alkyl C₁₋₆, (CH2)ₙNR₆R₇, in which R₆ and R₇, which may be the same or different represent H or alkyl C₁₋₆, (CH₂)ₘ-phenyl or a sugar, and n and m, which may be the same or different, represent an integer from 2-4 inclusive,
R₂ represents H or alkyl C1-6′
R₃ represents H or OCH₃,
R₄ represents OH or CH(CH₃)(CH₂)₃NH₂
and pharmaceutically acceptable salts thereof.

We prefer those compounds of formula III in which R¹ represents H or alkyl C₁₋₆, R² represents H and X₂ is N or O.

When R¹ in formula III represents a sugar, that sugar is preferably a hexose, such as galacotose, fructose or the sugar of example 34,

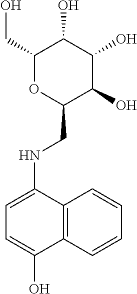

Also included within the scope of this invention are those compounds of formula III which may be dimerised on oxidation to give structures of the kind:

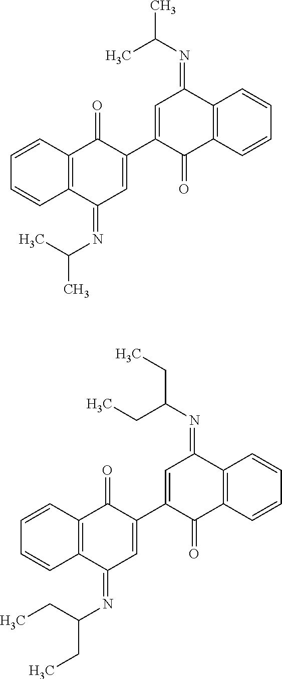

A preferred group of compounds of formula I are those of formula IV,

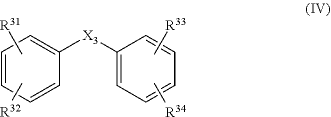

in which
X₃ represents CH₂, CO, NH, CH(OH), O;
R³¹, R³², R³³ and R³⁴, which may be the same or different, independently represent H, OH, Cl, NH₂ or CH₂OH;
in addition, R³¹ and R³³, when each in the 2 position with respect to X₃ may together form a single bond;
and pharmaceutically acceptable salts thereof.

Some of the compounds may already have had a pharmaceutical use described. Accordingly, we provided the use of the compounds of formula I, II, III and IV, and those compounds specifically exemplified herein, in the treatment of diseases in which inhibition of IDO plays a therapeutic role, particularly those conditions mentioned herein.

We particular prefer the compounds with the following structures:

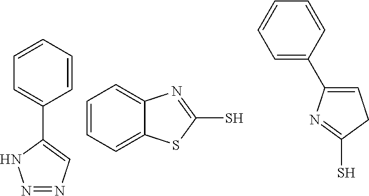

In the event that the description of compounds of formula covers compounds already known as the IDO inhibitors, then these compounds are specifically excluded from the scope of this invention.

The compounds of formula I may be used alone or in combination with at least one additional therapeutic agent.

The at least one additional therapeutic agent may be an antineoplastic chemotherapy agent. Suitable antineoplastic chemotherapeutic agent is selected from the group consisting of cyclophosphamide, methotrexate, fluorouracil, doxorubicin, vincristine, ifosfamide, cisplatin, gemcytabine, busulfan, ara-C, and combinations thereof.

Alternatively, the at least one additional therapeutic agent may be radiation therapy. The radiation therapy may be localized radiation therapy delivered to the tumour or may be total body irradiation.

The compounds of the invention may be used as an adjuvant to the therapeutic vaccination of various cancers. Cancers that may be mentioned include melanoma, colon cancer, pancreatic cancer, breast cancer, prostate cancer, lung cancer, leukemia, brain tumours, lymphoma, sarcoma, ovarian cancer, and Kaposi's sarcoma.

Other cancers and tumours that may be mentioned include adrenocorticocancer, basal cell carcinoma, bladder cancer, bowel cancer, brain and CNS tumors, breast cancers, B-cell lymphoma, carcinoid tumours, cervical cancer, childhood cancers, chondrosarcoma, choriocarcinoma, chronic myeloid leukemia, rectal cancers, endocrine cancers, endometrial cancer, esophageal cancer, Ewing's sarcoma, eye cancer, gastric cancer or carcinoma, gastrointestinal cancers, genitourinary cancers, glioma, gynecological cancers, head and neck cancers, hepatocellular cancer, Hodgkins disease, hypopharynx cancer, islet cell cancer, kidney cancer, laryngeal cancer, liver cancer, lung cancer (including small-cell lung carcinoma and non-small-cell carcinoma), lymphoma, male breast cancer, melanoma, mesothelioma, multiple myeloma, nasopharyngeal cancer, neuroblastoma, non-Hodgkins lymphoma, non-melanoma skin cancer, osteosarcoma, ovarian cancer, pancreas cancer, pituitary cancer, prostate cancer, renal cell carcinoma, retinoblastoma, rhabdomyosarcoma, sarcoma, skin cancer, squamous cell carcinoma, stomach cancer, testicular cancer or seminoma, thymus cancer, thyroid cancer, transitional cell cancer, trophoblastic cancer, uterine cancer, vaginal cancer, Waldenstrom's macroglobulinemia, and Wilm's tumor, colorectum, cervix, endometrium, ovary, testis, mesothelial lining, white blood cell (including lymphoma and leukemia) esophagus, muscle, connective tissue, adrenal gland, bone, glioblastoma, and cutaneous basocellular carcinoma.

In addition to cancers, IDO plays a role in several diseases, including Clamydia psittaci infection and Streptococcus pyogenes infection, systemic lupus erythematosus, rheumatoid arthritis, Alzheimer's disease, :Huntington's disease, Parkinson's disease, lyme neuroborreliosis, late lyme encephalopathy, Tourette's syndrome, systemic sclerosis, multiple sclerosis, coronary heart disease, T-cell mediated immune diseases, chronic infections (viral, bacterial, fungal and microbial), depression, neurological disorders, cancer tumors, and cataracts. Inhibitors of IDO may be used to treat these diseases.

Other diseases that IDO inhibitors may be used to treat include, but are not limited to, human immunodeficiency virus (HIV) and AIDS-related cancers.

The compounds may also be used as adjuvants to bone marrow transplantation or peripheral blood stem cell transplantation.

Where the compounds are used in the treatment of an infection, the infection may be selected from the group consisting of a viral infection, infection with an intracellular parasite, and infection with an intracellular bacteria.

Particular viral infections include human immunodeficiency virus or cytomegalovirus.

Particular intracellular parasite infections may be selected from the group consisting of Leishmania donovani, Leishmania tropica, Leishmania major. Leishmania aethiopica, Leishmania mexicana, Plasmodium falciparum, Plasmodium vivax, Plasmodium ovale, and Plasmodium malariae.

Particular intracellular bacterial infections may be selected from the group consisting of Mycobacterium leprae, Mycobacterium tuberculosis, Listeria monocytogenes, and Toxplasma gondii.

When the compounds are used in combination, the at least one additional therapeutic agent may be a vaccine, for example, an anti-viral vaccine, a vaccine against HIV, a vaccine against tuberculosis, a vaccine against malaria. The vaccine may also be a tumour vaccine or a melanoma vaccine. Preferably, the tumour vaccine comprises genetically modified tumour cells or genetically modified cell lines. In such cases, preferably the genetically modified tumour cells or genetically modified cell line has been transfected to express granulocyte-macrophage stimulating factor (GM-CSF).

Alternatively, the vaccine may comprise one or more immunogenic peptides, preferably immunogenic peptides of cancer-testis antigens (CTAgs). Such CTAgs and immunogenic peptides thereof are well known in the art, see Scanlan et al. Cancer Immun. 2004; 4:1 and Simpson et al., Nat Rev Cancer. 2005; 5:615. CTAg proteins include MAGE, BAGE, GAGE, SSX, NY-ESO-1, LAGE, SCP, CTSP, CT7, CT8, CT9, CT10, CT11, SAGE, OY-TES-1, NY-SAR-35 and NY-BR-1. Several MAGE proteins are known, including MAGE-A1, A3, A4, A5, A6, A8, A9, A10, A12, B1, B2, B3, B4, C1, C2 and C3 proteins. Several SSX proteins exist, including SSX1, SSX2, SSX3 and SSX5.

Further, the tumour vaccine may comprise dendritic cells.

Further, the additional therapeutic agent may be a cytokine, for example a granulocyte-macrophage colony stimulating factor (GM-CSF) or flt3-ligand.

According to the invention we further provide a method of treating a subject receiving a bone marrow transplant or peripheral blood stem cell transplant comprising administering a therapeutically effective amount of compound of formula I or a pharmaceutically acceptable salt thereof to such a subject.

Preferably, the compound of formula I or a pharmaceutically acceptable salt thereof is administered in an amount effective to increase the delayed type hypersensitivity reaction to tumour antigen, delay the time to relapse of post-transplant malignancy, increase relapse free survival time post-transplant, and/or increase long-term post-transplant survival.

Preferably, the compound of formula I or a pharmaceutically acceptable salt thereof is administered prior to full hematopoetic reconstitution.

Salts of compounds of formula I may be formed by reacting the free acid, or a salt thereof, with one or more equivalents of the appropriate base. The reaction may be carried out in a solvent or medium in which the salt is insoluble or in a solvent in which the salt is soluble, e.g. ethanol, tetrahydrofuran or diethyl ether, which may be removed in vacuo, or by freeze drying. The reaction may also be a metathetical process or it may be carried out on an ion exchange resin.

Pharmaceutically acceptable salts of compounds of formula I when it is an acid include alkali metal salts, e.g. sodium and potassium salts; alkaline earth metal salts, e.g. calcium and magnesium salts; salts of the Group III elements, e.g. aluminium salts; and ammonium salts. Salts with suitable organic bases, for example, salts with hydroxylamine; lower alkylamines, e.g. methylamine or ethylamine; with substituted lower alkylamines, e.g. hydroxy substituted alkylamines; or with monocyclic nitrogen heterocyclic compounds, e.g. piperidine or morpholine; and salts with amino acids, e.g. with arginine, lysine etc, or an N-alkyl derivative thereof; or with an aminosugar, e.g. N-methyl-D-glucamine or glucosamine. The non-toxic physiologically acceptable salts are preferred, although other salts are also useful, e.g. in isolating or purifying the product.

When the compound of formula I is a base, pharmaceutically acceptable salts thereof include salts with strong acids, e.g., HCl, HBr, etc, and salts with weak acids, eg organic acids, for example carboxylic acids, such as acetic acid, benzoic acids, as well as sulphonic acids.

The compounds of formula I or a pharmaceutically acceptable salt thereof for use in the method will generally be administered in the form of a pharmaceutical composition.

Thus, according to a further aspect of the invention there is provided a pharmaceutical composition including preferably less than 80% w/w, more preferably less than 50% w/w, e.g. 0.1 to 20%, of the compound of formula I or a pharmaceutically acceptable salt thereof, in admixture with a pharmaceutically acceptable diluent or carrier.

We also provide a process for the production of such a pharmaceutical composition which comprises mixing the ingredients. Examples of pharmaceutical formulations which may be used, and suitable diluents or carriers, are as follows:
for intravenous injection or infusion—purified water or saline solution;
for inhalation compositions—coarse lactose;
for tablets, capsules and dragees—microcrystalline cellulose, calcium phosphate, diatomaceous earth, a sugar such as lactose, dextrose or mannitol, talc, stearic acid, starch, sodium bicarbonate and/or gelatin;
for suppositories—natural or hardened oils or waxes.

When the compounds of formula I or a pharmaceutically acceptable salt thereof is to be used in aqueous solution, e.g. for infusion, it may be necessary to incorporate other excipients. In particular there may be mentioned chelating or sequestering agents, antioxidants, tonicity adjusting agents, pH-modifying agents and buffering agents.

Solutions containing the compound of formula I or a pharmaceutically acceptable salt thereof may, if desired, be evaporated, e.g. by freeze drying or spray drying, to give a solid composition, which may be reconstituted prior to use.

When not in solution, the compound of formula I or a pharmaceutically acceptable salt thereof preferably is in a form having a mass median diameter of from 0.01 to 10 μm. The compositions may also contain suitable preserving, stabilising and wetting agents, solubilisers, e.g. a water-soluble cellulose polymer such as hydroxypropyl methylcellulose, or a water-soluble glycol such as propylene glycol, sweetening and colouring agents and flavourings. Where appropriate, the compositions may be formulated in sustained release form.

The content of the compound of formula I or a pharmaceutically acceptable salt thereof in a pharmaceutical composition is generally about 0.01-about 99.9 wt %, preferably about 0.1-about 50 wt %, relative to the entire preparation.

The dose of the compound of formula I or a pharmaceutically acceptable salt thereof is determined in consideration of age, body weight, general health condition, diet, administration time, administration method, clearance rate, combination of drugs, the level of disease for which the patient is under treatment then, and other factors.

While the dose varies depending on the target disease, condition, subject of administration, administration method and the like, for oral administration as a therapeutic agent for the treatment of cancer in a patient suffering from such a disease is from 0.01 mg-10 g, preferably 0.1-100 mg, is preferably administered in a single dose or in 2 or 3 portions per day. Where the compound of formula I or a pharmaceutically acceptable salt thereof is used in combination with other therapeutic agents, these may be used at their normal therapeutic doses, e.g., as set out in pharmacopoeias or prescribing guides, such as the Physicians' Desk Reference (PDR). In certain cases, the compound of formula I or a pharmaceutically acceptable salt thereof supplements the activity of the additional therapeutic agent(s) in a synergistic fashion, such that the additional therapeutic agent(s) can be administered at a lower dose than is normally used.

The potential activity of the compound of formula I or a pharmaceutically acceptable salt thereof in the treatment of cancer or infections has been demonstrated in the following predictive experiments, which demonstrate that the compound of formula I is an IDO inhibitor.

### Experimental Methods

The enzymatic inhibition assays were performed as described by Takikawa et al. [Takikawa, O.; Kuroiwa, T.; Yamazaki, F.; Kido, R. J Biol Chem, 1988, 263, 2041-2048.] with some modifications. Briefly, the reaction mixture (100 μl) contained potassium phosphate buffer (100 mM, pH 6.5) ascorbic acid (20 mM), catalase (200 units/ml), methylene blue (10 μM), purified recombinant IDO (2 ng/μl), and L-Trp (200 μM). The inhibitors were serially diluted ranging from 0.1 to 1000 μM. The reaction was carried out at 37° C. for 60 min and stopped by the addition of 30% (w/v) trichloroacetic acid (40 μl). To convert the product of Trp dioxygenation by IDO, N-formylkynurenine, to spectroscopically detectable kynurenine, the tubes were incubated at 50° C. for 30 min, followed by a centrifugation at 10000 g for 20 min. Lastly, 100 μl of supernatant from each probe was transfered to another tube for HPLC analysis. The mobile phase for HPLC measurements consisted of 50% sodium citrate buffer (40 mM, pH 2.25) and 50% methanol with 400 μM SDS. The flow rate through the S5-ODS1 column was chosen to be 1 ml/min, and kynurenine was detected at a wavelength of 365 nm.

### CHEMISTRY

### Preparation of 4-aminoalkyl-1-naphtol Derivatives Through Reductive Amination

4-amino-1-naphthol is commercially available compound and exists as hydrochloric salt. 4-amino-1-naphthol have been treated by triethylamine in dichloromethan, then followed by addition of t-Butyldimethylsilyl trifluoromethanesulfonate at 0° C. to protect the phenol selectively as t-Butyldimethylsilyl group, we found latter that this step is not necessary and we can apply the reductive amination reaction on free protected compound. 4-amino-1-naphthol hydrochloride have been suspended in dichloroethane then treated with acetic acid, and different aldehydes and ketones, then followed by slow addition of sodium triacetoxyborohydride, the mixture then have been stirred over night. After the sodium bicarbonate work up and chromatography, products from 1 to 4 have been obtained in 70, 94, 82, 92% yield respectively, then followed by preparation the corresponding hydrochloric salt by treating the amines by dry hydrochloric acid in iso-propanol or dioxane in diethyl ether as solvent, the formed precipitate have been filtrate and the solid have been washed by diethyl ether, the desire salt from 1′-4′ have been obtained in pure form.

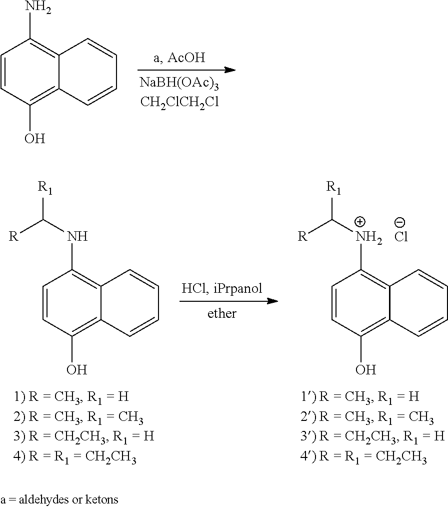

### Preparation of 4-aminoalkyl-1-naphtol Derivatives Through Nucluphilic Substitution

p-Naphthohydroquinone have been dissolved in toluene, then little excess of amine have been added, the mixture have been refluxed between one hour, then after chromatography on silica gel, products from 5 to 9 have been obtained in 65, 84, 95, 77, 89% yield respectively, then followed by preparation the corresponding hydrochloric salt by treating the amines by dry hydrochloric acid in iso-propanol or dioxane in diethyl ether as solvent, the formed precipitate have been filtrate and the solid have been washed by diethyl ether, the desire salt from 5′-9′ have been obtained in pure form.

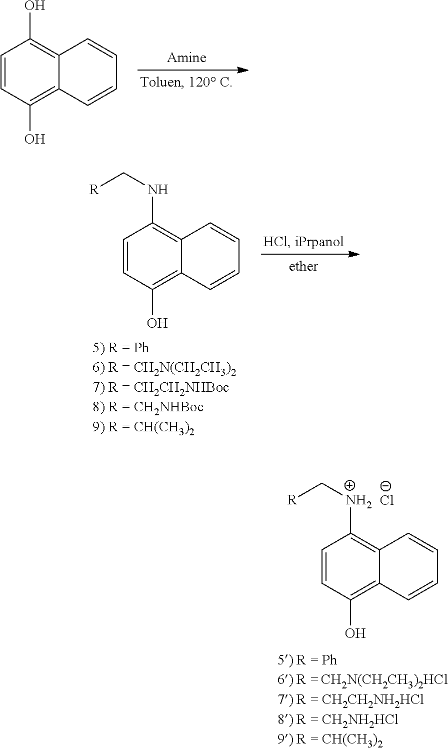

### Preparation of 5-Aminoalkyl-8-hydroxyquinoline Derivatives Through Reductive Amination

5-Amino-8-hydroxyquinoline dihydrochloride have been suspended in dichloroethane then treated with acetic acid, and different acetaldehyde or acetone, then followed by slow addition of sodium triacetoxyborohydride, the mixture then have been stirred over night. After the sodium bicarbonate work up and chromatography, products from 10 and 11 have been obtained in 81, 96% yield respectively, then followed by preparation the corresponding dihydrochloric salt by treating the amines with dry hydrochloric acid in iso-propanol or dioxane in diethyl ether as solvent, the formed precipitate have been filtrate and the solid have been washed by diethyl ether, the desire salt of 10′ and 11′ have been obtained in pure form.

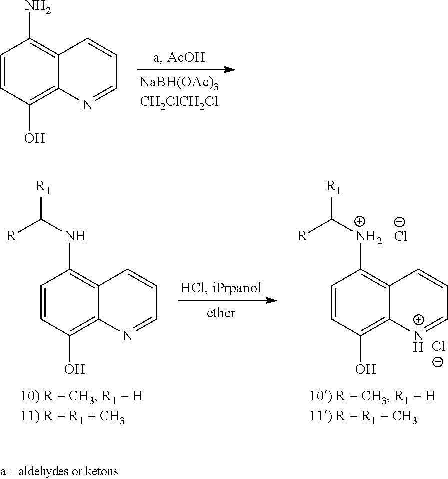

### Preparation of 4-amino(aminoacids)-1-naphtol Derivatives Through Peptide Coupling

4-amino-1-naphthol hydrochloride have been suspended in dichloroethane then amino acid have been added acid, by slow addition of N,N-Diisopropylethylamine, the mixture have been stirred for 15 minutes, then a mixture of HATU, HOAt, DIEA in DCM have been added, the mixture have been stirred for 1 h after the work up, products from 12 to 16 have been obtained in 90, 94, 93, 85, 83% yield respectively, then followed by Boc deprotection and preparation the corresponding hydrochloric salt by treating the aminoacid derevative by dry hydrochloric acid in isopropanol or dioxane in diethyl ether as solvent, the formed precipitate have been filtrate and the solid have been washed by diethyl ether, the desire salt from 12′-16′ have been obtained in pure form.

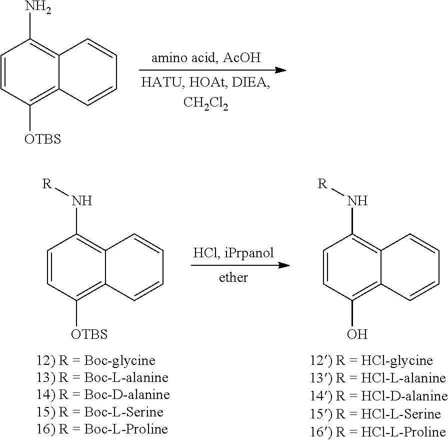

### Preparation of Alkyl Triazoles Derivatives Through Click Chemistry

Different aryl alkynes have been prepared by applying special method of click chemistry, the mixture of alkyne, sodium azide, sodium ascorbate, copper sulphate in dioxane have been stirred at 80° C. for 6 hours, then after the work up, the crud product have been dissolved in water followed by addition of 3M aqueous sodium hydroxide, after 24 hours the desired triazoles 17, 18, 19 have been obtained in 98, 93, 81% yields respectively. The hydrochloric salts of these triazole have been obtained by treating the amines by dry hydrochloric acid in iso-propanol or dioxane in diethyl ether as solvent, the formed precipitate have been filtrate and the solid have been washed by diethyl ether, the desired salt from 17′, 18′, 19′ have been obtained in pure form.

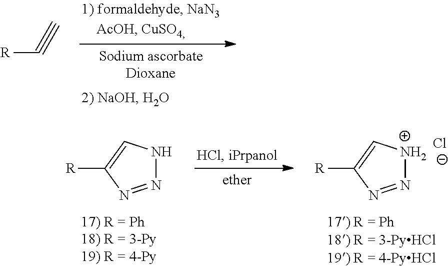

Different alkynes have been reacted with p-azidophenol under above click chemistry conditions to give substituted triazole ring 20, 21, 22 in 84, 65, 93% yield respectively. The corresponding salts of these analogues have been prepared as before.

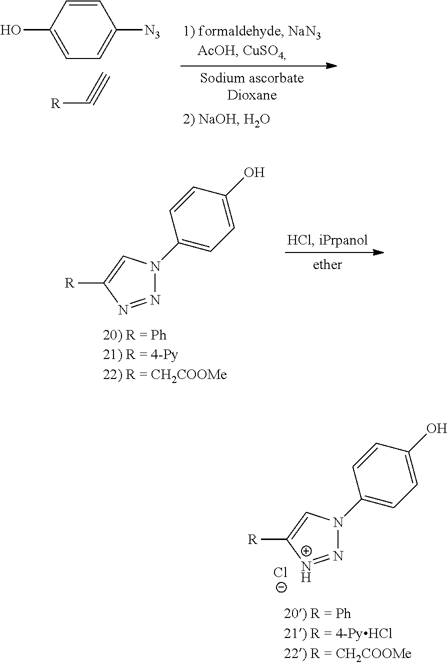

### Synthesis of the Oxidized Form of Alkylamines 2, and 4

Alkyl amines 2, 4 have been dissolved in a mixture of ether water mixture, then aqueous 1 M NaOH, followed by excess of H₂O₂, at 0° C., the reaction have been stirred for 2 hours, we obtained dimeric form when we applied these conditions on product 2, and 4. The new dimeric analogues 23, and 24 have been obtained in 45, and 56% yield respectively, these analogues show excellent activity in vitro.

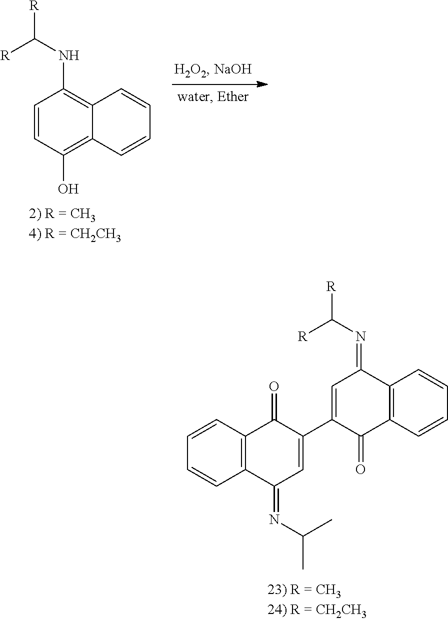

### Structures of Examples 1-54, and IC₅₀ Data as IDO Inhibitors in Enzymatic Assay

### Example 1

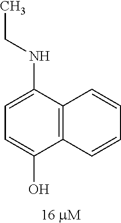

### Example 2

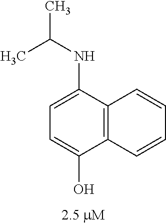

### Example 3

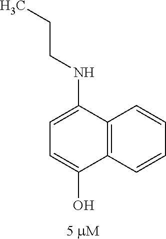

### Example 4

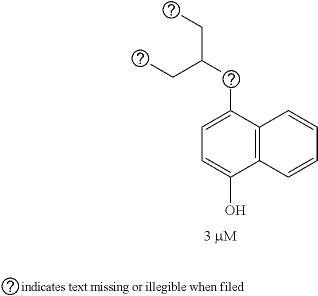

### Example 5

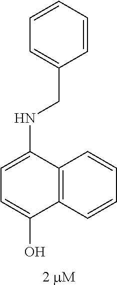

### Example 6

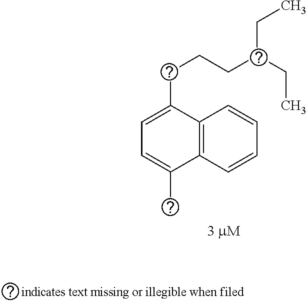

### Example 7

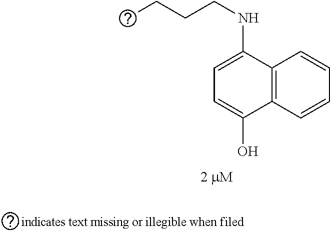

### Example 8

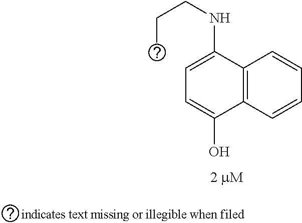

### Example 9

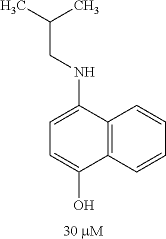

### Example 10

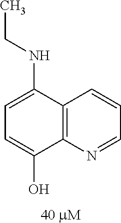

### Example 11

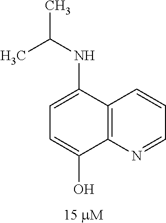

### Example 12

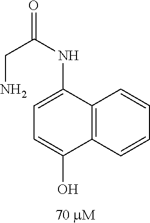

### Example 13

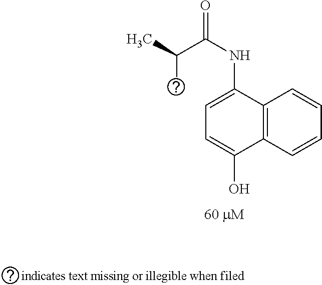

### Example 14

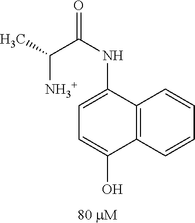

### Example 15

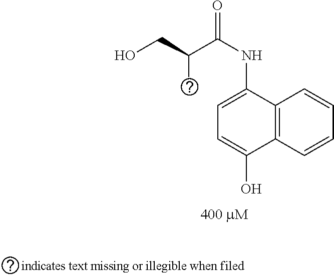

### Example 16

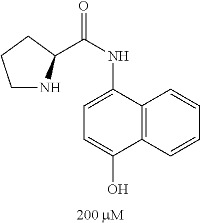

### Example 17

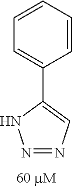

### Example 18

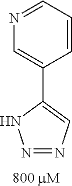

### Example 19

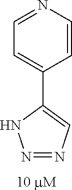

### Example 20

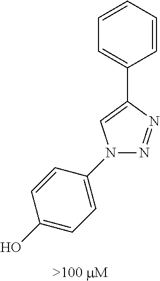

### Example 21

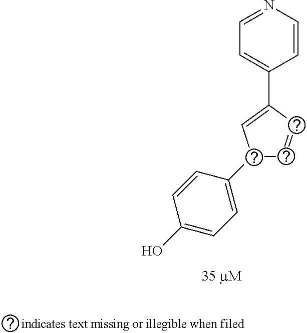

### Example 22

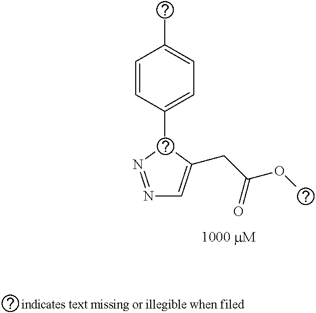

### Example 23

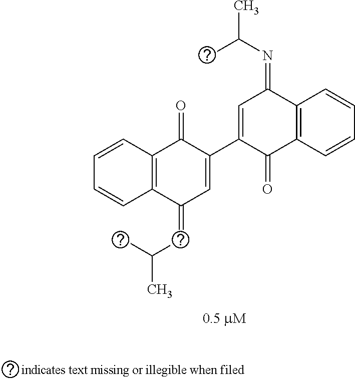

### Example 24

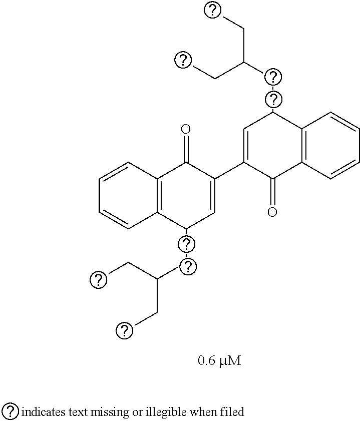

### Example 25

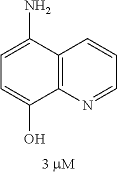

### Example 26

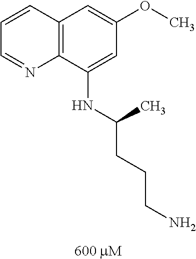

### Example 27

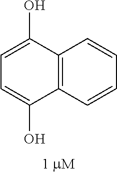

### Example 28

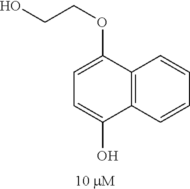

### Example 29

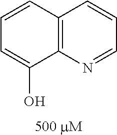

### Example 30

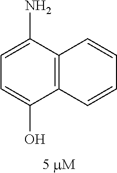

### Example 31

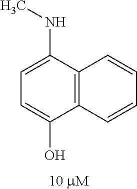

### Example 32

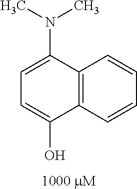

### Example 33

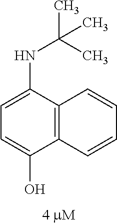

### Example 34

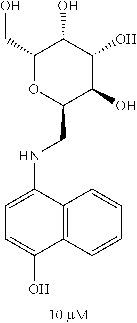

### Example 35

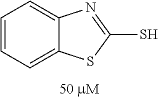

### Example 36

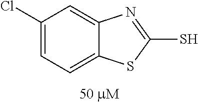

### Example 37

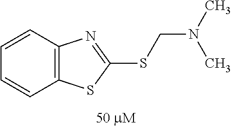

### Example 38

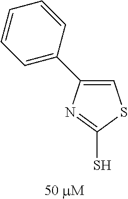

### Example 39

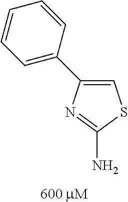

### Example 40

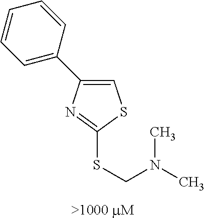

### Example 41

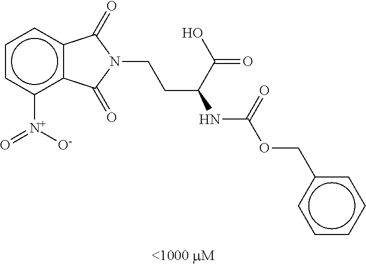

### Example 42

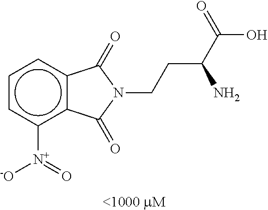

### Example 43

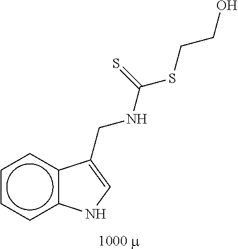

### Example 44

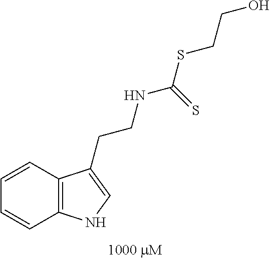

### Example 45

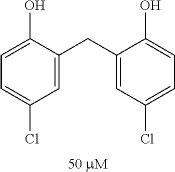

### Example 46

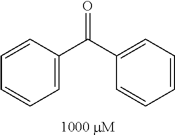

### Example 47

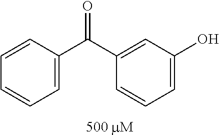

### Example 48

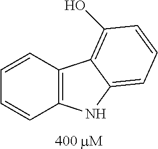

### Example 49

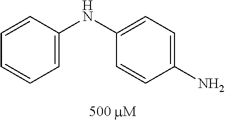

### Example 50

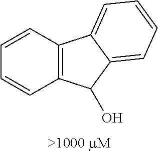

### Example 51

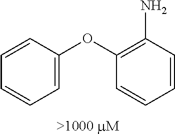

### Example 52

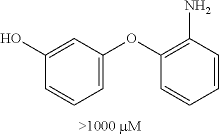

### Example 53

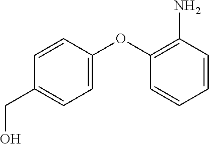

### Example 54

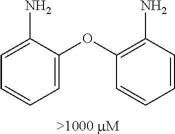

1-14. (canceled)

15. A compound which binds to human indoleamine 2,3 dioxygenase (IDO) having formula: 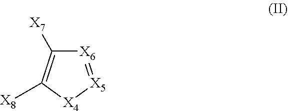
in which
X₄ represents NR¹¹ or S, wherein R¹¹ represents H, pyridyl or phenyl optionally substituted by —OH;
X₅ represents N or CR¹², wherein R¹² represents H, NH₂ or SR₁₃ and R₁₃ represents H or CH₂N(CH₃)₂;
X₆ represents N or CR¹⁴, wherein R¹⁴ represents H or (CH₂)ₚNHC(S)S(CH₂)_{q}OH in which p and q, which may be the same or different, represent an integer from 1-4;
either one of X₇ and X₈ represents pyridyl, CH₂C(O)OCH₃ or phenyl optionally substituted by —OH, and the other of X₇ and X₈ represents H, or
X₇ and X₈, together with the carbon atoms to which they are attached form a benzene ring which is optionally substituted by NO₂ or chlorine,
and pharmaceutically acceptable salts thereof.

16. The compound of claim 15, wherein X₄ is NR¹¹, and both X₅ and X₆ are N.

17. The compound of claim 15, wherein X₄ is S, X₅ is C—R¹² and X₆ is N.

18. The compound of claim 17, wherein R₁₂ is SH, NH₂, or CH₂N(CH₃)₂.

19. The compound of claim 15, wherein X₄ is NH, X₅ is CH, and X₆ is R¹⁴.

20. The compound of claim 19, wherein R¹⁴ is (CH₂)ₚNHC(S)S(CH₂)₂OH wherein p is 1 or 2.

21. the compound of claim 15, of formula

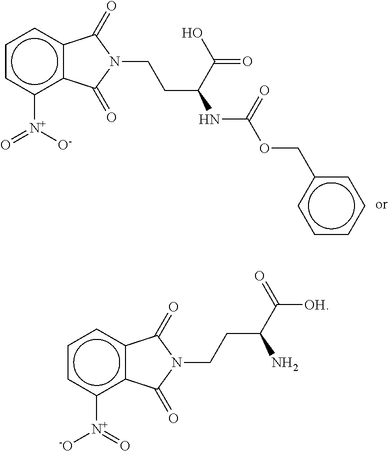

22. The compound of claim 15, having formula: 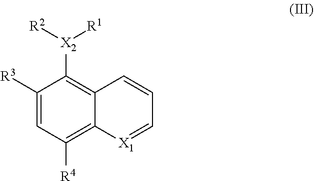
in which
X₁ represents N or C,
X₂ represents H, N or O, provided that when X2 represents H, R1 and R2 have no value and that when X₂ represents O, R₂ has no value,
R₁ represents H, alkyl C₁₋₆, (CH₂)ₙNR₆R₇, in which R₆ and R₇, which may be the same or different represent H or alkyl C₁₋₆, (CH₂)ₘ-phenyl or a sugar, and n and m, which may be the same or different, represent an integer from 2-4 inclusive,
R₂ represents H or alkyl C1-6,
R₃ represents H or OCH₃,
R₄ represents OH or CH(CH₃)(CH₂)₃NH₂
and pharmaceutically acceptable salts thereof.

23. The compound of claim 22, wherein R¹ is H or C₁-C₆ alkyl, branched or unbranched, R² is H, and X₂ is N or O.

24. The compound of claim 22, wherein R₁ is a hexose.

25. The compound of claim 24, wherein said hexose is galactose, fructose, or

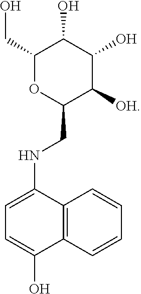

26. The compound of claim 22, of formula

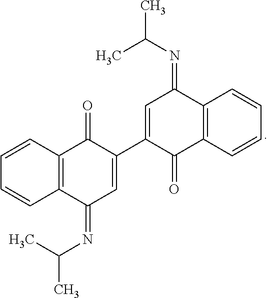

27. The compound of claim 15, of formula 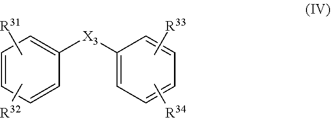
in which
X₃ represents CH₂, CO, NH, CH(OH), O;
R³¹, R³², R³³ and R³⁴, which may be the same or different, independently represent H, OH, Cl, NH₂ or CH₂OH;
in addition, R³¹ and R³³, when each in the 2 position with respect to X₃ may together form a single bond;
and pharmaceutically acceptable salts thereof.
